(19) United States
(12) Patent Application Publication
(10) Pub. No.: US 2017/0253541 A1
(43) Pub. Date: Sep. 7, 2017

(54) METHOD AND APPARATUS FOR PRODUCING HYDROCARBONS

(71) Applicant: Linde Aktiengesellschaft, München (DE)

(72) Inventors: Torben Höfel, München (DE); Helmut Fritz, München (DE)

(21) Appl. No.: 15/510,346

(22) PCT Filed: Sep. 11, 2015
(86) PCT No.: PCT/EP2015/070893

§ 371 Date: Mar. 10, 2017

(30) Foreign Application Priority Data
Sep. 11, 2014 (EP) 14184470.4

(51) Int. Cl. C07C 1/20; C07C 5/25; B01J 19/24; C07C 4/02

(52) U.S. Cl. CPC C07C 1/20; C07C 4/02; C07C 5/25; B01J 19/245; B01J 2219/24

(57) ABSTRACT

A method for producing hydrocarbons is proposed wherein a catalysis product stream (b) rich in n-butane, isobutane, 1-butene, 2-butene, isobutene and hydrocarbons with more than four and/or less than four carbon atoms is produced in a catalysis unit (1), using one or more catalyst feed streams containing oxygenates and/or olefins (a) and wherein additionally a steam cracking product stream (h) is produced in a steam cracking unit (2) using one or more steam cracking feed streams (g, r, s). It is provided that using the catalysis product stream (b) a skeletal isomerisation feed stream (f, q) poor in 1-butene, 2-butene and isobutene and containing at least isobutane is produced, in which the isobutane is at least predominantly reacted by skeletal isomerisation to form n-butane, and which is subsequently used at least partly as the, or one of the, steam cracking feed streams (g, r). The invention also relates to an apparatus (100, 200).

The invention relates to a method and an apparatus for producing hydrocarbons according to the pre-characterising clauses of the respective independent claims.

### PRIOR ART

Short-chain olefins such as ethylene and propylene can be produced by steam cracking hydrocarbons. Methods and apparatus for steam cracking hydrocarbons are described for example in the article “Ethylene” in Ullmann's Encyclopedia of Industrial Chemistry, online edition, 15 Apr. 2007, DOI 10.1002/14356007.a10_045.pub2.

Alternative methods of obtaining short-chain olefins are the so-called oxygenate-to-olefin methods (OTO). In oxygenate-to-olefin methods, oxygenates such as methanol or dimethyl ether are introduced into a reaction zone of a reactor in which a catalyst suitable for converting the oxygenates has been provided. The oxygenates are converted into ethylene and propylene, for example. The catalysts and reaction conditions used in oxygenate-to-olefin methods are basically known to the skilled man.

Oxygenate-to-olefin methods may be carried out with different catalysts. For example, zeolites such as ZSM-5 or SAPO-34 or functionally comparable materials may be used. If ZSM-5 or a comparable material is used, comparatively large amounts of longer-chained (C3plus) hydrocarbons (for designation see below) and comparatively small amounts of shorter-chained (C2minus) hydrocarbons are formed. When SAPO-34 or comparable materials are used, by contrast, shorter-chained (C2minus) hydrocarbons tend to be formed.

Integrated methods and apparatus (combined apparatus) for producing hydrocarbons which comprise steam cracking processes and oxygenate-to-olefin processes or comprise corresponding cracking furnaces and reactors are known and are described for example in WO 2011/057975 A2 or US 2013/0172627 A1.

Integrated methods of this kind are advantageous, for example, because typically not only the desired short-chain olefins are formed in the oxygenate-to-olefin processes. A substantial proportion of the oxygenates is converted into paraffins and C4plus olefins. At the same time, in steam cracking, not all the furnace feed is cracked into short-chain olefins. In particular, as yet unreacted paraffins may be present in the cracked gas of corresponding cracking furnaces. Moreover, C4plus olefins including diolefins such as butadiene are typically found here. The compounds obtained depend in both cases on the feeds and reaction conditions used.

In the methods proposed in W02011/057975 A2 and US 2013/0172627 A1 the cracked gas of a cracking furnace and the discharge stream from an oxygenate-to-olefin reactor are combined in a joint separating unit and fractionated. After hydrogenation or separation of butadiene, for example, a C4 fraction obtained here may again be subjected to a steam cracking process and/or an oxygenate-to-olefin process. The C4 fraction may be separated into predominantly olefinic and predominantly paraffinic partial fractions.

The present invention is not restricted to oxygenate-to-olefin processes but may basically be used with any desired catalytic methods, particularly catalytic methods in which the zeolites described hereinbefore are used as catalysts. Besides methanol and/or dimethyl ether, other oxygenates, for example, other alcohols and/or ethers, may be used as the feed in corresponding catalytic processes.

Similarly, olefinic components such as, for example, a mixture of different unsaturated C4 hydrocarbons, may be used in corresponding catalytic processes. In this case, the term olefin cracking process (OCP) is used. Different feeds may be introduced into the same reactor or different reactors within the scope of the present invention. For example, an oxygenate-to-olefin process may be carried out in one reactor and an olefin cracking process in another reactor. Both processes, and optionally also a combined process, have the objective, however, of producing a product which is rich in propylene and optionally ethylene from one or more feeds.

The catalytic processes described, which are characterised in that, in particular, the zeolites mentioned are used as catalysts and moreover one or more catalyst feed streams containing oxygenates and/or olefin are used, are thus carried out in a catalysis unit which may contain one or more corresponding reactors.

As already mentioned, the aim of catalytic processes of this kind is to produce products which are rich in propylene and optionally ethylene. Typically, however, in such processes, significant amounts of hydrocarbons with four or more carbon atoms are produced. It is therefore known from the prior art to recycle such hydrocarbons having four or more carbon atoms to the catalysis. It is also known to remove such hydrocarbons as product(s). It has also previously been proposed to subject the hydrocarbons having four or more hydrocarbon atoms or parts or fractions thereof to further reaction processes.

However, all the known processes have drawbacks. In particular, the efficiency of the utilisation of corresponding hydrocarbons in such processes is often unsatisfactory.

There is therefore a need for improvements to such processes and apparatus for the production of hydrocarbons using the catalytic methods described.

### DISCLOSURE OF THE INVENTION

This problem is solved by a method and an apparatus for producing hydrocarbons having the features of the independent claims. Preferred embodiments are the subject of the dependent claims and the description that follows.

Before the explanation of the features and advantages of the present invention, their basis as well as the terminology used will be explained.

Liquid and gaseous streams may, in the terminology as used herein, be rich in or poor in one or more components, “rich” indicating a content of at least 75%, 90%, 95%, 99%, 99.5%, 99.9% or 99.99% and “poor” indicating a content of at most 25%, 10%, 5%, 1%, 0.1% or 0.01% on a molar, weight or volume basis. The term “predominantly” may correspond to the definition of “rich” provided above, but refers in particular to a content or proportion of more than 90%. Liquid and gaseous streams may also, in the terminology used herein, be enriched or depleted in one or more components, these terms also applying to a corresponding content in a starting mixture from which the liquid or gaseous stream was obtained. The liquid or gaseous stream is “enriched” if it contains at least 1.1 times, 1.5 times, 2 times, 5 times, 10 times, 100 times or 1,000 times the amount, “depleted” if it contains at most 0.9 times, 0.5 times, 0.1 times, 0.01 times or 0.001 times the amount of a corresponding component, based on the starting mixture.

A liquid or gaseous stream is “derived” or “formed” from another liquid or gaseous stream (which is also referred to as the starting stream) if it comprises at least some components that were present in the starting stream or obtained therefrom. A stream derived or formed in this way may be obtained from the starting stream particularly by separating off or deriving a partial stream or one or more components, concentrating or depleting one or more components, chemically or physically reacting one or more components, heating, cooling, pressurising and the like.

Current methods for separating product streams from processes for preparing hydrocarbons include the formation of a number of fractions based on the different boiling points of the components present. In the art, abbreviations are used for these which indicate the carbon number of the hydrocarbons that are predominantly or exclusively present. Thus, a “C1 fraction” is a fraction which predominantly or exclusively contains methane (and by convention also contains hydrogen in some cases, and is then also called a “C1minus fraction”). A “C2 fraction” on the other hand predominantly or exclusively contains ethane, ethylene and/or acetylene. A “C3 fraction” predominantly contains propane, propylene, methylacetylene and/or propadiene. A “C4 fraction” predominantly or exclusively contains butane, butene, butadiene and/or butyne, while the respective isomers may be present in different amounts depending on the source of the C4 fraction. The same also applies to the “C5 fraction” and the higher fractions. Several such fractions may also be combined. For example, a “C2plus fraction” predominantly or exclusively contains hydrocarbons with two or more carbon atoms and a “C2minus fraction” predominantly or exclusively contains hydrocarbons with one or two carbon atoms.

By oxygenates are typically meant ethers and alcohols. Besides methyl tert. butyl ether (MTBE), it is also possible to use, for example, tert. amyl methyl ether (TAME), tert. amyl ethyl ether (TAEE), ethyl tert. butyl ether (ETBE) and diisopropyl ether (DIPE). Alcohols which may be used include for example methanol, ethanol and tert. butanol (TBA, tertiary butyl alcohol) . The oxygenates also include, in particular, dimethyl ether (DME, dimethyl ether). The invention is also suitable for use with other oxygenates.

According to a common definition which is also used here, oxygenates are compounds which comprise at least one alkyl group covalently bonded to an oxygen atom. The at least one alkyl group may comprise up to five, up to four or up to three carbon atoms. In particular, the oxygenates which are of interest within the scope of the present invention comprise alkyl groups with one or two carbon atoms, particularly methyl groups. Of particular interest are monohydric alcohols and dialkyl ethers such as methanol and dimethyl ether or corresponding mixtures thereof.

Steam cracking processes are carried out on a commercial scale almost exclusively in tubular reactors in which individual reaction tubes (in the form of coiled tubes, so-called coils) or groups of corresponding reaction tubes can be operated even under different cracking conditions. Reaction tubes or sets of reaction tubes operated under identical or comparable cracking conditions and possibly also tube reactors operated under uniform cracking conditions are also referred to as “cracking furnaces”. A cracking furnace in the terminology used here is thus a construction unit used for steam cracking which subjects a furnace feed to identical or comparable cracking conditions. A steam cracking unit used within the scope of the present invention may comprise one or more cracking furnaces of this kind.

The same also applies, as already mentioned, to the catalysis unit used within the scope of the present invention, in which different reactors can be provided with identical or different catalysts, supplied with identical or different feed streams and operated under identical or different reaction conditions.

The term “steam cracking feed stream” here refers to one or more liquid and/or gaseous streams which are supplied to one or more cracking furnaces. Streams obtained by a corresponding steam cracking process, as described hereinafter, may also be recycled into one or more cracking furnaces and thus used again as steam cracking feed streams. Suitable steam cracking feed streams include a number of hydrocarbons and hydrocarbon mixtures from ethane to gas oil up to a boiling point of typically 600° C.

A steam cracking feed stream may thus exclusively comprise so-called “fresh feed”, i.e. a feed which is prepared outside the apparatus and is obtained for example from one or more petroleum fractions, petroleum gas and/or petroleum gas condensates. A steam cracking feed stream may, however, also additionally or exclusively comprise one or more so-called “recycle streams”, i.e. streams that are produced in the apparatus itself and recycled into a corresponding cracking furnace. A steam cracking feed stream may also consist of a mixture of one or more fresh feeds with one or more recycle streams.

The steam cracking feed stream is at least partly reacted in the cracking furnace and leaves it as a so-called “crude gas” which can be subjected to after-treatment steps. These encompass, first of all, processing of the crude gas, for example by quenching, cooling and drying, so as to obtain a “cracked gas”. Occasionally the crude gas is also referred to as cracked gas. In the present case, the term “steam cracking product stream” is used for this.

Again, the same also applies to the feed stream or streams supplied to one or more catalysis units, which are referred to here as “catalysis feed streams”. The catalysis feed stream or streams are reacted in the catalysis unit in one or more reactors to form one or more product streams referred to here as “catalysis product streams”.

In more recent steam cracking processes and apparatus, mild cracking conditions are increasingly used, as they enable particularly so-called value products such as propylene to be obtained in larger amounts. Basically, processes in which the cracking conditions are adapted to the composition of the steam cracking feed streams are advantageous. Under mild conditions, however, the reaction in the cracking furnace or furnaces is also reduced, so that unreacted compounds are found in comparatively large amounts in the cracking product stream or streams and thus lead to a “dilution” of the value products that are to be recovered.

The “cracking conditions” in a cracking furnace mentioned above encompass inter alia the partial pressure of the furnace feed, which may be influenced by the addition of different amounts of steam and the pressure selected in the cracking furnace, the dwell time in the cracking furnace and the temperatures and temperature profiles used therein. The furnace geometry and configuration also play a part.

As the values mentioned influence one another at least partially, the term “cracking severity” has been adopted to characterise the cracking conditions. For liquid furnace feeds, the cracking severity can be described by means of the ratio of propylene to ethylene (P/E) or as the ratio of methane to propylene (M/P) in the cracked gas, based on weight (kg/kg). For gaseous furnace feeds, by contrast, the reaction or conversion of a particular component of the furnace feed can be stated as a measure of the cracking severity. In particular for hydrocarbons with four carbon atoms, the cracking severity can usefully be described by means of the reaction of key components such as n-butane and isobutane. For a technical understanding of the term “cracking severity”, reference may be made to the previously mentioned article “Ethylene” in Ullmann's Encyclopedia of Industrial Chemistry.

### ADVANTAGES OF THE INVENTION

The present invention combines the measures described hereinbefore for making optimum use of hydrocarbons with four carbon atoms from a corresponding catalysis process, so as to achieve efficient utilisation with maximum extraction of value and minimal internal recycle streams.

For this purpose the present invention starts from a method for the production of hydrocarbons, which comprises producing a catalysis product stream containing n-butane, isobutane, 1-butene, 2-butene, isobutene and hydrocarbons with more than four and/or less than four carbon atoms, in a catalysis unit using one or more catalysis feed streams containing oxygenates and/or olefins. The catalysis unit comprises, as previously stated, one or more reactors which are supplied with one or more feed streams, referred to here as catalysis feed streams.

As explained, the present invention is suitable for use with oxygenate-to-olefin processes and/or the so-called olefin cracking processes and other processes. The reactor or reactors used in a corresponding catalysis unit preferably comprise zeolites as catalysts. As explained, these catalysts may be of the SAPO or ZSM type, in particular. The catalysis unit used within the scope of the present invention is thus set up for a corresponding catalysis process.

The method further provides that a steam cracking product stream be produced in a steam cracking unit using one or more steam cracking feed streams. The steam cracking process used within the scope of the present invention may be carried out in one or more cracking furnaces, using identical or different steam cracking conditions, as is fundamentally known. For details, see the above explanations. In particular, the steam cracking feed streams used in the steam cracking may be cracked under mild conditions in order to increase the yield of value products. More severe cracking conditions may be used in particular to achieve the highest possible conversion.

According to the invention it is now provided that, using the catalysis product stream, a skeletal isomerisation feed stream is produced which is poor in 1-butene, 2-butene and isobutene and contains at least isobutane, in which the isobutane is at least predominantly reacted by skeletal isomerisation to form n-butane, and which is then used at least partly as the or one of the steam cracking feed streams.

Thus, an essential aspect of the present invention is the use of skeletal isomerisation for processing branched hydrocarbons with four carbon atoms from a steam cracking process. As a result, predominantly unbranched components are subjected to the steam cracking process, resulting in an increased conversion and improved selectivity towards the desired target products ethylene and propylene.

As will also be explained in more detail hereinafter, the skeletal isomerisation feed stream poor in 1-butene, 2-butene and isobutene and containing at least isobutane can also be produced by, inter alia, reacting any isobutene still present in the steam cracking product stream by hydrogenation to form isobutane. The latter can then be subjected to skeletal isomerisation in the skeletal isomerisation feed stream. In particular, because of its thermal stability, isobutene is highly unsuitable for use in a steam cracking process and therefore cannot be used to good effect in conventional processes. A corresponding skeletal isomerisation feed stream may be obtained by inter alia hydrogenating all the olefins obtained. If desired, it is also possible to obtain the skeletal isomerisation feed stream by distillation, optionally prior to which a hydroisomerisation from 1-butene to 2-butene is performed.

Where there is a mention here, and in the following description, of “at least predominantly” removing, processing, separating or reacting one or more hydrocarbons, this may encompass, as mentioned in the introduction, the removal of at least 75%, 90% or more of such hydrocarbons. Preferably, corresponding hydrocarbons are removed substantially completely, i.e. in particular by at least 95%, optionally by at least 99% or more.

The method proposed within the scope of the present invention is particularly efficient as it also makes use of isobutene, which is inherently unsuitable for steam cracking, in the form of isobutane, which is subsequently reacted to form n-butane, and hence can be fed into the steam cracking like all the other components as a steam cracking feed stream or steam cracking streams. In this way it is also possible to maximise the quantity of butadiene which is formed in the steam cracking, as, in the absence of isobutene, the cracking conditions can be largely adapted to ensure maximum butadiene production.

One essential difference, within the scope of the present invention, from a known method, as disclosed for example in US 2013/0172627 A1, is that within the scope of the present invention paraffinic and olefinic components, including isobutene after suitable reaction, can be fed into the steam cracking and the process results in more than just separation into C4 olefins and C4 paraffins.

Some embodiments of the invention which have already been partly discussed and which are recited in the dependent claims will be further summarised hereinafter.

In particular, the process according to the invention envisages that, using at least part of the catalysis product stream, a separation feed stream should be formed from which the hydrocarbons with more than four and/or less than four carbon atoms are at least predominantly separated, to obtain a separation discharge stream rich in n-butane, isobutane, 1-butene, 2-butene and isobutene. Besides the catalysis product stream or part of it, theoretically a further stream, for example the steam cracking product stream, or at least part of it, may also be fed into a corresponding separation feed stream, which may thus be processed in the same separator as the catalysis product stream or a corresponding part thereof. This once again results in improved integration of a catalytic process, as explained hereinbefore, and of a steam cracking process. The hydrocarbons with more than four and/or less than four carbon atoms which are at least predominantly separated from the separation feed stream may be separated once more in the form of individual fractions, prepared as products, and/or recycled into the catalysis unit and/or the steam cracking unit. Details are not described here, in the interests of clarity.

Particularly advantageously, in a process according to the invention, using at least part of the separation discharge stream, which, as already mentioned, is rich in n-butane, isobutane, 1-butene, 2-butene and isobutene and contains few, or no, hydrocarbons with more than four and/or less than four carbon atoms, a hydrogenation feed stream is formed in which the 1-butene, 2-butene and isobutane are reacted, at least predominantly by hydrogenation, to form n-butane and isobutene, thereby forming a hydrogenation discharge stream. Where there is a mention here, or hereinafter, of a corresponding stream being “formed”, this may also merely encompass the use of a corresponding stream which does not necessarily have to be processed as described above. At least part of the hydrogenation discharge stream obtained may be used in the formation of the skeletal isomerisation feed stream. As already explained, all or just part of the hydrogenation discharge stream may thus be used as a skeletal isomerisation feed stream.

It is particularly advantageous if, using at least part of the separation discharge stream, a distillation feed stream is formed from which the n-butane and 2-butene are at least predominantly removed, to obtain a distillation discharge stream which is poor in n-butane and 2-butene. Such distillative separation makes it easier to carry out the subsequent processing of the distillation discharge stream, from which, as explained hereinafter, or using which, the skeletal isomerisation feed stream is produced. In skeletal isomerisation or in the steps preceding it, the volume flows to be dealt with are significantly reduced, thanks to suitable distillation.

A particularly advantageous process in which corresponding distillation is used comprises using at least part of the separation discharge stream to form a hydroisomerisation feed stream wherein the 1-butene is reacted at least predominantly by hydroisomerisation into 2-butene, producing a hydroisomerisation discharge stream. At least part of the hydroisomerisation discharge stream can then be used during the formation of the distillation feed stream. Corresponding hydroisomerisation significantly facilitates the separation of isobutene, which is subsequently subjected, as already mentioned, to hydrogenation and skeletal isomerisation, from the linear butenes which do not necessarily have to be subjected to a corresponding treatment: The boiling point of isobutene at atmospheric pressure is −6.9° C., while that of 1-butene is −6.47° C. Thus, distillative separation is practically impossible. The boiling point of the 2-butenes, by contrast, is significantly higher than this, namely 3.7° C. for cis-2-butene and 0.9° C. for trans-2-butene.

Using at least part of the distillation discharge stream from a distillation process as described previously, advantageously a hydrogenation feed stream is formed in which at least the isobutane is reacted at least predominantly by hydrogenation to form isobutene, thereby producing a hydrogenation discharge stream. The hydrogenation discharge stream obtained is used at least partly in the formation of the skeletal isomerisation feed stream in which the isobutene obtained from the isobutene by the hydrogenation can be reacted particularly easily by skeletal isomerisation.

If a distillative process is used, as explained previously, advantageously a stream is formed, using at least some of the n-butane and 2-butene separated from the distillation feed stream, which can be used as a further steam cracking feed stream. The further steam cracking feed stream may be fed into the same cracking furnace or a different cracking furnace than the steam cracking feed stream described previously which has been formed from, or using, the skeletal isomerisation feed stream.

In the steam cracking process or the steam cracking unit, a steam cracking product stream is formed which contains hydrocarbons with four carbon atoms, including butadiene, as well as hydrocarbons with more than four and/or less than four carbon atoms. As already explained, a corresponding stream may also be processed in a separating unit associated with the catalysis unit.

Advantageously, using at least part of the steam cracking product stream, a residual stream is obtained which is poor in butadiene and hydrocarbons with more than four and/or less than four carbon atoms, which is used at least partly in the production of the skeletal isomerisation feed stream. In particular, such a residual stream may be subjected for example to hydrogenation prior to distillative separation or hydroisomerisation.

The invention also relates to an apparatus which is designed for the production of hydrocarbons. Such an apparatus comprises a catalysis unit which is configured to produce a catalysis product stream rich in n-butane, isobutane, 1-butene, 2-butene, isobutene and hydrocarbons with more than four and/or less than four carbon atoms, using one or more catalyst feed streams containing oxygenates and/or olefins, as well as a steam cracking unit which is configured to produce a steam cracking product stream, using one or more steam cracking feed streams.

An apparatus of this kind is characterised by means which are configured to produce, using the catalysis product stream, a skeletal isomerisation feed stream poor in 1-butene, 2-butene and isobutene and containing at least isobutane, to react the isobutane therein at least predominantly by skeletal isomerisation to obtain n-butane, and then to use the latter at least partly as the, or one of the, steam cracking feed streams.

An apparatus of this kind which particularly comprises means configured to carry out a process as described hereinbefore benefits from the advantages described previously, to which reference will therefore be specifically made.

Methods of hydrogenation, skeletal isomerisation and hydroisomerisation, as used within the scope of the present invention, are known in principle to the skilled man.

Skeletal isomerisation may be carried out, for example, using aluminium oxide catalysts (in which y-aluminium oxide may be used as an adsorbent, as a catalyst support and/or as the catalyst itself). Activated and/or steam-treated aluminium oxide may also be used, for example, as stated in U.S. Pat. No. 3,558,733. In addition, compounds containing titanium or boron may be used, particularly in conjunction with q- or y-aluminium oxide, as described in U.S. Pat. No. 5,321,195 and U.S. Pat. No. 5,659,104. Other compounds which may be used include halogenated aluminium oxides, as disclosed for example in U.S. Pat. No. 2,417,647, bauxites or zeolites. It is also known to use molecular sieves of microporous structure, as known for example from EP 0 523 838 A1, EP 0 501 577 A1 and EP 0 740 957 A1. The latter may also form active phases of catalysts.

Aluminium oxide-based catalysts are generally used in the presence of water at temperatures of 200 to 700° C. and pressures of 0.1 to 2 MPa, particularly at temperatures of 300 to 570° C. and pressures of 0.1 to 1 MPa. Other reaction conditions for skeletal isomerisation may be inferred from the publications mentioned.

Numerous catalytic methods for the hydrogenation and hydroisomerisation of olefins or olefin-containing hydrocarbon mixtures are known from the prior art, and may also be used within the scope of the present invention. Hydrogenation catalysts comprise, as hydrogenation-active components, one or more elements of the 6^{th}, 7^{th} or 8^{th} subgroup of the Periodic Table, in elemental or bound form. Typically, noble metals of the 8^{th} subgroup are used as hydroisomerisation catalysts. They may be doped with various additives in order to influence specific catalyst properties such as service life, resistance to certain catalyst poisons, selectivity or regenerability. The hydrogenation and hydroisomerisation catalysts contain the active component in many forms on carriers, for example mordenites, zeolites, Al₂O₃ modifications, SiO₂ modifications, etc.

Hydroisomerisation processes are described for example in EP 1 871 730 B1, US 2002/169346 A1, U.S. Pat. No. 6,420,619 B1, U.S. Pat. No. 6,075,173 and WO 93/21137 A1. In processes of this kind, typically a corresponding stream is passed through a hydroisomerisation reactor in the presence of a hydroisomerisation catalyst. The hydroisomerisation reactor is typically embodied as a fixed bed reactor. Preferably, the hydroisomerisation process results in the 1-butene being extensively reacted to form 2-butene. However, the reaction that is actually carried out will depend inter alia on economic considerations.

Generally, reaction temperatures of 150 to 250° C. are used for the extensive hydrogenation of the olefins, while hydroisomerisation is carried out at significantly lower temperatures. The thermodynamic equilibrium is weighted towards the internal olefins, in this case 2-butenes, at these lower temperatures.

The invention and preferred embodiments of the invention will be described hereinafter with reference to the appended drawings, which show preferred embodiments of the invention.

### BRIEF DESCRIPTION OF THE DRAWINGS

FIG. 1 shows an apparatus according to one embodiment of the invention, in schematic representation.

FIG. 2 shows an apparatus according to one embodiment of the invention, in schematic representation.

### EMBODIMENTS OF THE INVENTION

The Figures show corresponding elements with identical reference numerals and are not repeatedly explained, in the interests of clarity. The streams shown in the respective Figures are given identical reference numerals when they have essentially the same or a comparable composition, irrespective of any differences in volume flows. In all the Figures, a catalysis unit is designated 1 and a steam cracking unit is designated 2.

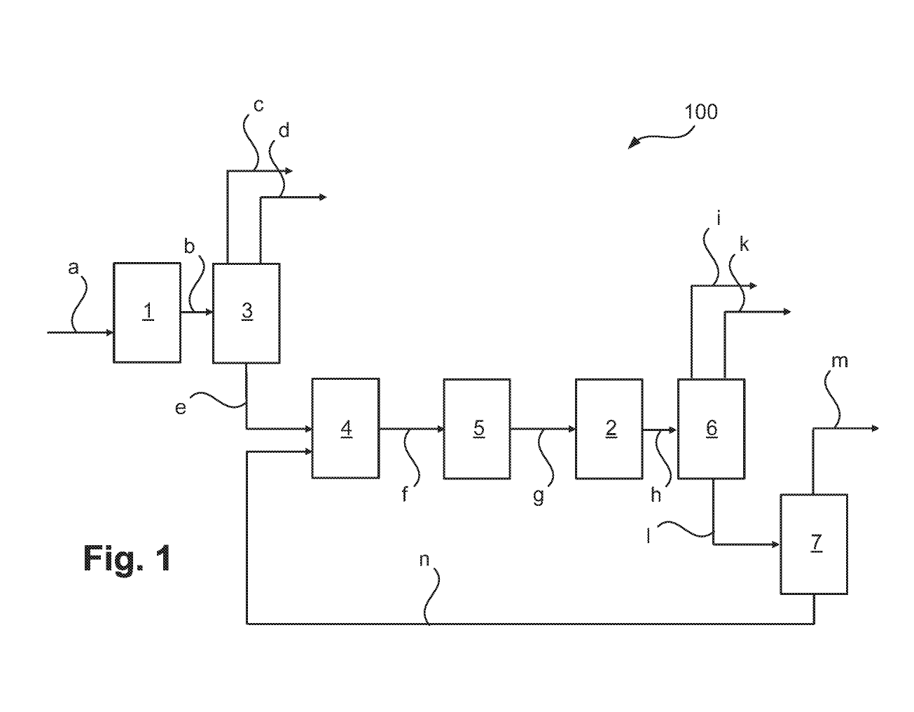

In FIG. 1 an apparatus according to one embodiment of the invention is shown schematically in simplified view and is generally designated 100.

One or more catalysis feed streams, here designated a, containing oxygenates and/or olefins are supplied to the catalysis unit 1. As already mentioned, the catalysis unit 1 may comprise one or more reactors which are operated with a zeolite catalyst. The catalysis unit may additionally be supplied with further streams, not shown here.

In the embodiment shown a catalysis product stream b is produced in the catalysis unit 1. It is fed as a separation feed stream to a separating unit 3, in which a stream e depleted in hydrocarbons with more than four and/or less than four carbon atoms or rich in hydrocarbons with four carbon atoms, referred to here as the separation discharge stream, is obtained from the catalysis product stream b. The streams separated off, here designated c and d, may for example comprise hydrocarbons with five or more and/or hydrocarbons with three or less carbon atoms, or other such fractions. Streams of this kind may also be processed in a corresponding apparatus and/or obtained as products.

In the embodiment shown, the separation discharge stream e is combined with another stream n described hereinafter, referred to here as a residual stream, thereby producing a hydrogenation feed stream which is fed to a hydrogenation unit 4. In the hydrogenation unit 4, preferably all the unsaturated hydrocarbons of streams e and n are reacted to form corresponding saturated hydrocarbons. However, partial hydrogenation is also possible. In the hydrogenation unit 4 a stream is obtained which is designated the hydrogenation discharge stream.

Whereas the separation discharge stream e in the embodiment shown typically still contains all the hydrocarbons with four carbon atoms which are produced in the catalysis unit 1, or originate from the catalysis feed stream(s) a and have not been reacted in the catalysis unit 1, particularly n-butane, isobutane, 1-butene, 2-butene and isobutene, the hydrogenation discharge stream still contains only, or predominantly, the corresponding saturated hydrocarbons, i.e. n-butane and isobutane.

In the embodiment shown the hydrogenation discharge stream is fed as a skeletal isomerisation feed stream f to an isomerisation unit 5 in which the isobutane contained in the skeletal isomerisation feed stream f is reacted to form n-butane. A skeletal isomerisation discharge stream obtained in the skeletal isomerisation unit 5 therefore predominantly or exclusively contains n-butane and is fed into the steam cracking unit 2 as a steam cracking feed stream g.

The steam cracking feed stream g, i.e. essentially pure n-butane, for example, is processed in the steam cracking unit 2 in one or more cracking furnaces, optionally also together with further streams which are fed into the same or different cracking furnaces. A steam cracking product stream h is obtained which, as already explained, contains hydrocarbons with four carbon atoms, including butadiene, as well as hydrocarbons with more than four and/or less than four carbon atoms. This steam cracking product stream h is fed into a further separating unit 6, in which, initially, by separating off hydrocarbons with more than four and/or less than four carbon atoms, as illustrated here by the streams i and k, a stream I is obtained which predominantly contains hydrocarbons with four carbon atoms, including butadiene. In the embodiment shown the stream I is fed into a butadiene separating unit 7, where the butadiene present is predominantly separated off and discharged from the apparatus as a stream m. A remaining residual stream, here designated n, which is low in butadiene can be combined with the above-mentioned hydrogenation feed stream or the separation discharge stream e which forms it and fed into the hydrogenation unit 4.

Depending on the desired result, severe cracking (to maximise ethylene) or mild cracking (to maximise propylene) can be carried out in the steam cracking unit 2.

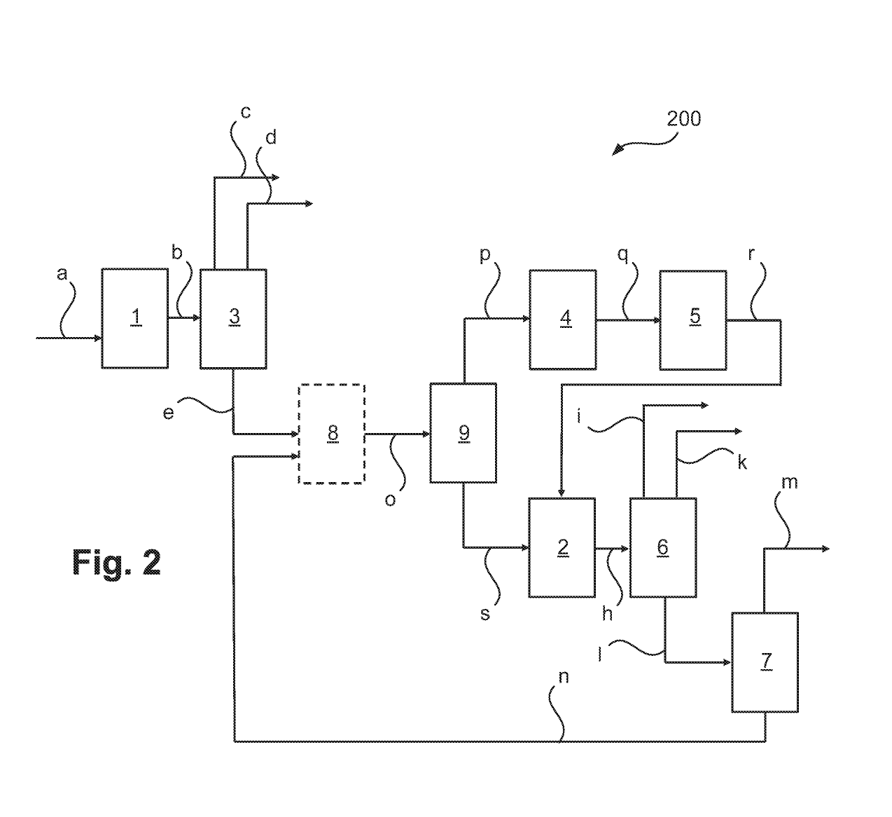

However, irrespective of the cracking severity, there is a tendency for a larger amount of butadiene to be produced only when the feed used still contains unsaturated components, namely 1-butene and/or 2-butene, in particular (which is not the case in the example of the apparatus 100 in the form of the steam cracking feed stream g). If a larger amount of butadiene is required, an apparatus 200 according to FIG. 2 may be used.

FIG. 2 schematically shows an apparatus according to another embodiment of the invention, generally designated 200.

In the apparatus 200, a separation discharge stream e from the separating unit 3 is optionally supplied to a hydroisomerisation unit 8. To do this, a combined stream, also referred to here as a hydroisomerisation feed stream, may be formed from the separation discharge stream e and the above-mentioned residual stream n low in butadiene. Instead of being fed into the hydroisomerisation unit 8 the separation discharge stream e and the residual stream n may also be fed directly into a distillation unit 9 in the form of a distillation feed stream o, which means that the hydroisomerisation unit 8 is optional. If a hydroisomerisation unit 8 is used, the 1-butene contained in the hydroisomerisation feed stream or the separation discharge stream e and the residual stream n is reacted therein to form 2-butene, which is contained in a corresponding hydroisomerisation discharge stream or the distillation feed stream o formed therefrom. As already explained, this assists with distillative separation in the distillation unit 9.

In the distillation unit 9, a distillation discharge stream p is obtained from the distillation feed stream o by separating off butane and 2-butene, or the major part thereof, in the form of the stream s. If there is no hydroisomerisation unit 8 the distillation discharge stream p contains 1-butene, but otherwise it does not. In addition, the distillation discharge stream p contains isobutene and isobutane. The distillation discharge stream p is supplied as hydrogenation feed stream to a hydrogenation unit, designated 4 here, as in FIG. 1. In the hydrogenation unit, 1-butene is reacted to 1-butane, if present in the hydrogenation feed stream p, and additionally isobutene is reacted to form isobutane. A hydrogenation discharge stream obtained in the hydrogenation unit 4 therefore contains either a mixture of essentially n-butane and isobutane or essentially pure isobutane. The hydrogenation discharge stream is then fed as a skeletal isomerisation feed stream q to a skeletal isomerisation unit, which is also designated 5 here. Once again, a substantially pure n-butane stream is obtained as the skeletal isomerisation discharge stream and is fed, as the steam cracking feed stream r, to the steam cracking unit 2.

In the distillation unit 9 a stream essentially containing butane and 2-butene is also obtained, as described, which can also be supplied as a (further) steam cracking feed stream s to a steam cracking unit, and can be fed into the same or a different cracking furnace from the steam cracking feed stream r.

Regarding the streams a to d and h to n and the units 1 to 3 and 6 and 7, reference is made to the foregoing remarks on FIG. 1.

1. Method for producing hydrocarbons, wherein a catalysis product stream (b) rich in n-butane, isobutane, 1-butene, 2-butene, isobutene and hydrocarbons with more than four and/or less than four carbon atoms is produced in a catalysis unit (1), using one or more catalyst feed streams containing oxygenates and/or olefins (a), and wherein additionally a steam cracking product stream (h) is produced in a steam cracking unit (2) using one or more steam cracking feed streams (g, r, s), characterised in that using the catalysis product stream (b) a skeletal isomerisation feed stream (f, q) poor in 1-butene, 2-butene and isobutene and containing at least isobutane is produced, in which the isobutane is at least predominantly reacted by skeletal isomerisation to form n-butane, and which is subsequently used at least partly as the, or one of the, steam cracking feed streams (g, r).

2. Method according to claim 1, wherein, using at least part of the catalysis product stream (b), a separation feed stream is formed from which the hydrocarbons with more than four and/or less than four carbon atoms are at least predominantly separated off, to obtain a separation discharge stream (e) rich in n-butane, isobutane, 1-butene, 2-butene and isobutene.

3. Method according to claim 2, wherein, using at least part of the separation discharge stream (e), a hydrogenation feed stream is formed in which the 1-butene, 2-butene and isobutene are reacted at least predominantly by hydrogenation, to form n-butane and isobutane, thereby producing a hydrogenation discharge stream, at least part of the hydrogenation discharge stream being used in the formation of the skeletal isomerisation feed stream (f).

4. Method according to claim 2, wherein, using at least part of the separation discharge stream (e), a distillation feed stream (o) is formed, from which the n-butane and 2-butene are at least predominantly separated off, thereby forming a distillation discharge stream (p) which is poor in n-butane and 2-butene.

5. Method according to claim 4, wherein, using at least part of the separation discharge stream (e), a hydroisomerisation feed stream is formed in which the 1-butene is reacted at least predominantly by hydroisomerisation to form 2-butene, thereby producing a hydroisomerisation discharge stream, at least part of the hydroisomerisation discharge stream being used in the formation of the distillation feed stream (o).

6. Method according to claim 4 or 5, wherein, using at least part of the distillation discharge stream (p), a hydrogenation feed stream is formed wherein at least the isobutene is reacted at least predominantly by hydrogenation to form isobutane, thereby producing a hydrogenation discharge stream, at least part of the hydrogenation discharge stream being used in the formation of the skeletal isomerisation feed stream (q).

7. Method according to one of claims 4 to 6, wherein, using at least part of the n-butane and 2-butene separated from the distillation feed stream (o), a stream is formed which is used as a further steam cracking feed stream (s).

8. Method according to one of the preceding claims, wherein the steam cracking product stream (h) contains hydrocarbons with four carbon atoms, including butadiene, as well as hydrocarbons with more than four and/or less than four carbon atoms.

9. Method according to claim 8, wherein, using at least part of the steam cracking product stream (h), a residual stream (n) which is poor in butadiene and hydrocarbons with more than four and/or less than four carbon atoms is obtained, which is at least partly used in the production of the skeletal isomerisation feed stream (f, q).

10. Apparatus (100, 200) for the production of hydrocarbons, having a catalysis unit (1) which is configured to produce a catalysis product stream (b) rich in n-butane, isobutane, 1-butene, 2-butene, isobutene and hydrocarbons with more than four and/or less than four carbon atoms, using one or more catalyst feed streams containing oxygenates and/or olefins (a), and having a steam cracking unit (2) which is configured to produce a steam cracking product stream (h), using one or more steam cracking feed streams (g, r, s), characterised by means which are configured to produce a skeletal isomerisation feed stream (f, q) poor in 1-butene, 2-butene and isobutene and containing at least isobutane, using the catalysis product stream (b), to react the isobutane therein at least predominantly by skeletal isomerisation to form n-butane, and then to use the latter at least partly as the, or one of the, steam cracking feed streams (g, r).

11. Apparatus (100, 200) according to claim 10, which comprises means configured for carrying out a method according to one of claims 1 to 9.
